(19) United States
(12) Patent Application Publication
(10) Pub. No.: US 2026/0053746 A1
(43) Pub. Date: Feb. 26, 2026

(54) COMPOSITIONS AND METHODS OF USE THEREOF FOR PREVENTION OF MASTITIS

(71) Applicant: CureMast, Inc., Atlanta, GA (US)

(72) Inventor: Thomas Harry Barrows, Austell, GA (US)

(21) Appl. No.: 19/306,334

(22) Filed: Aug. 21, 2025

Related U.S. Application Data
(60) Provisional application No. 63/685,517, filed on Aug. 21, 2024.

(51) Int. Cl. A61K 9/1274; A61K 9/00; A61K 9/107; A61K 9/51; A61P 29/00

(52) U.S. Cl. CPC A61K 9/1274; A61K 9/0041; A61K 9/107; A61K 9/5123; A61K 9/5146; A61P 29/00

(57) ABSTRACT

Disclosed herein are compositions and methods of use thereof for the prevention of mastitis in dairy cattle. The compositions and methods can have immunostimulant effectiveness against mastitis-causing bacteria. In particular embodiments, the methods include preventing mastitis infection by intramammary infusion of a disclosed composition at dry-off.

### CROSS-REFERENCED TO RELATED APPLICATIONS

This application claims the benefit of and priority to U.S. Provisional Application No. 63/685,517, filed Aug. 21, 2024, which is hereby incorporated herein by reference in its entirety.

### FIELD OF THE INVENTION

The technical field of the invention generally includes formulations, typically immunostimulant formulations, for prevention and treatment of mastitis, particularly bovine mastitis.

### BACKGROUND OF THE INVENTION

Dairy farming is an important contribution to food supply worldwide. Although the reliable production of safe and wholesome milk is commonly viewed as proven and secure, this is the result of constant attention to the health of dairy cattle and insurance that milk from infected cows is discarded. Despite knowledge of the best practices for control, mastitis is a pervasive problem and the most common and costly disease in the dairy industry. The economic loss due to mastitis in the United States alone is estimated to be about 2 billion dollars per year, about 70% due to decreased milk production. A discussion of the background on bovine mastitis is provided by Lopez, et. al in WO2014/053977, as referenced herein.

Mastitis prevention with the use of good sanitation, antiseptic teat dips, and optimal feed diets is extremely important. Another common and highly recommended practice is the intramammary infusion of an antibiotic via the teat canal of each quarter for each cow in the herd at the end of its lactation cycle, known as the “dry-off” period.

Nevertheless, cows still suffer from mastitis at a troublesome rate. Typically, about 20% of the herd will have a low-grade, sub-clinical infection that causes no outward signs of distress but reduces milk production and results in the shedding of white blood cells into the milk. If the co-called “somatic cell count” (SCC) exceeds a certain value, the milk cannot be sold and the cow contributing to excessive SCC test results of milk in the bulk tank must be separated from the herd until cured. The cure rate is very low because antibiotics infused into the mammary of a lactating cow get washed out during milking, which must be continued 2 to 3 times each day. Typically, at least a third of cows severely infected during lactation end up being “culled”, meaning that they are prematurely sold for slaughter. Culling is not profitable and represents a significant cost of dairy farming.

Improved means of preventing mastitis is needed. As shown in FIG. 1, a point in the lactation cycle having a high incidence of new mastitis infections is at dry-off. During this period the immune system of the mammary is overburdened in the process of absorbing milk that is not being milked out and bacteria that enter the teat encounter a weakened immune response. Until recently it was common practice to infuse a dose of antibiotic into all four teats of each cow at dry-off regardless of her infection status. This practice is now being criticized as an example of the overuse of antibiotics in the livestock industry, and can lead to the emergence of new, antibiotic-resistant bacteria. Unfortunately, there remains no alternative for prevention of mastitis other than such indiscriminate use of antibiotics.

The immunotherapy approach to preventing mastitis is currently focused on vaccine development. However, this “adaptive immunity” approach is limited in its need to target specific bacteria and has not yielded a universally applicable, cost-effective product.

Providing an alternative to antibiotics used in the dairy industry will also benefit human health. It is well recognized that indiscriminate and overuse of antibiotics has accelerated the emergence of antibiotic-resistant bacteria. More than 2.8 million antibiotic-resistant infections occur in the U.S. each year, and more than 35,000 people die as a result (CDC 2019). Special attention is being given to the use of antibiotics in livestock that are important for use in preventing and treating infections in humans. All antibiotics used to treat mastitis are classified by the World Health Organization as either important or critically important for use in humans (Ruegg 2020). Critically important antibiotics on that list include amoxicillin and ampicillin.

There remains a need for convenient and economical compositions and methods of reducing severity of, or preventing, mastitis, particularly mastitis in cows and/or goats, that are safe and effective.

Thus, it is an object of the invention to provide compositions and methods of use thereof for the reduction of severity of, or prevention of, mastitis.

It is another object of the invention to reduce the amount, and/or frequency of use, of antibiotics in the treatment of mastitis, and thus preferably reduce overuse, ineffectiveness, and potential emergence of antibiotic-resistant strains of mastitis-causing bacteria.

### SUMMARY OF THE INVENTION

Compositions and methods for the treatment and prevention of mastitis in animals, particularly dairy animals, including but not limited to, cows and goats, are provided. The compositions typically include an emulsion of lipid particles, preferably nanoparticles, known as “cubosomes”.

Cubosomes have the basic molecular architecture of liposomes, namely an aqueous compartment surrounded by a lipid bilayer, except that the aqueous and lipid phases in cubosomes are further organized into interpenetrating, bicontinuous regions. This unique liquid crystalline structure sets cubosomes apart from other nanoparticles and offers several advantages for use as vehicles for drug delivery. Exemplary lipid substances that are useful in forming cubosomes include, but are not limited to, monoolein, phosphatidyl choline, and phytantriol.

Methods of treating animals, particularly cows and goats, most particularly dairy cows and goats, to reduce the severity of, or prevent mastitis, are also provided. The methods can include infusing any of the disclosed compositions into the mammary gland, for example, via the teat canal.

### BRIEF DESCRIPTION OF THE FIGURES

FIG. 1 illustrates the incidence of intramammary infection during the lactation cycle (adapted from Bradley 2004).

FIG. 2 illustrates a cubosome exhibiting its cavernous internal and cubic structure and its membrane composition with different drug loading modalities (adapted from Bhosale 2013).

FIG. 3 is a photograph of a sample of resolidified “cubic gel”.

FIG. 4 is a cryo-TEM image of an emulsion of dispersed cubosomes prepared from cubic gel.

FIG. 5 is an illustration of the treatment plan for a study in 4 cows in which the cubosome formulation 59R was compared to other test materials and controls to determine its effectiveness in preventing mastitis infection.

FIG. 6A is a plot of the bacteria counts in milk secretions taken from cows after dry-off according to the treatment plan of FIG. 5.

FIG. 6B illustrates the results of the treatment plan of FIG. 5.

FIG. 7A is a line graph showing somatic cell count (SCC) in milk from quarters treated with FT1 (76R), FT2 (59R), or saline at dry-off, with or without subsequent E. coli challenge, measured over time relative to dry-off. The timing of E. coli infusion is indicated.

FIG. 7B are representative images of milk collected 24 h after dry-off from quarters receiving FT1+E. coli, FT2+E. coli, or saline+E. coli.

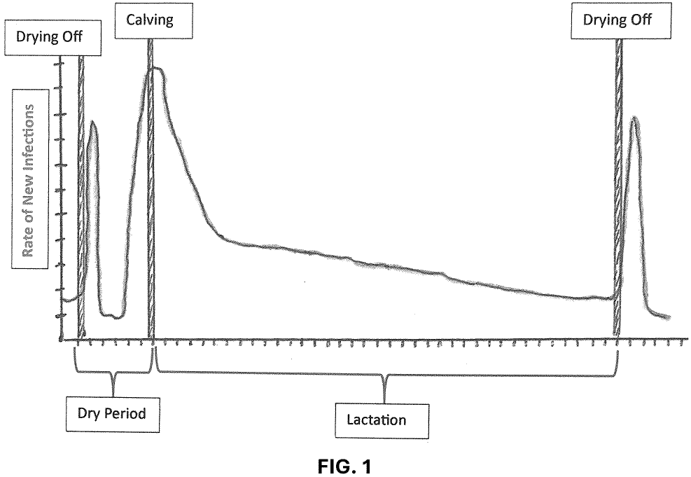

### DETAILED DESCRIPTION OF THE INVENTION

### I. Definitions

As used herein, the term “dry-off” means cessation of milking at the end of the lactation cycle, typically 2 months before the next parturition or dry period when the cow's mammary glands will need to undergo involution and regeneration in preparation for the next lactation cycle.

As used herein, the term “dry-cow therapy” comprises intramammary administration of a composition (such as antibiotics) after the last milking of the lactation cycle (i.e. at dry-off). The objective of dry-cow therapy is to clear existing infections and prevent new infections in the early dry period.

As used herein, the term “nanoparticle” generally refers to a particle having a diameter from about 1 nanometer (nm) up to, but not including, about 1 micron (1,000 nm), or from about 50 nm to about 500 nm. The particles can have any shape.

As used herein, the term “cubosome” means a nanoparticle that is cubic rather than spherical and has an internal, grid-like morphology visible in cryogenic transmission electron microscopy (“cryo-TEM”) images, as exemplified in FIG. 4.

As used herein, the term “cubic gel” means a solid or semisolid cubosome precursor substance that exhibits birefringence.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D.

Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as herein can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Although the description of materials, compositions, components, steps, techniques, etc. can include numerous options and alternatives, this should not be construed as, and is not an admission that, such options and alternatives are equivalent to each other or, in particular, are obvious alternatives.

### II. Compositions

The provided compositions (interchangeably referred to as formulations) typically include an aqueous emulsion of cubosomes formed by the comminution and dispersion of cubic gel including an amphiphilic lipid, water, and a surfactant stabilizer.

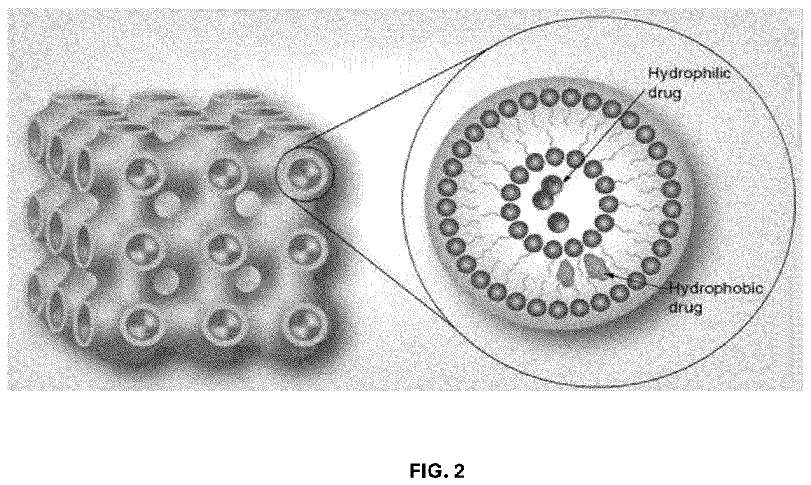

Cubosomes have the basic molecular architecture of liposomes, namely an aqueous compartment surrounded by a lipid bilayer, except that the aqueous and lipid phases in cubosomes are further organized into interpenetrating, bicontinuous regions. This unique liquid crystalline structure, illustrated in FIG. 2, sets cubosomes apart from other nanoparticles and offers several advantages for use as vehicles for drug delivery.

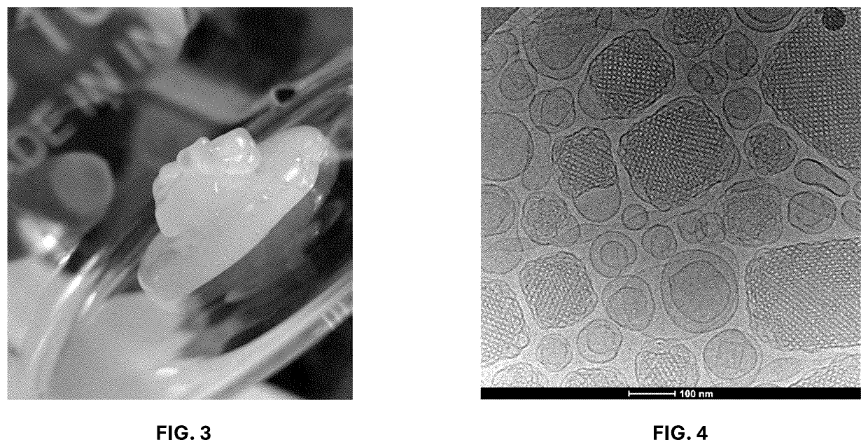

Cubosomes are composed of an amphiphilic lipid and a stabilizer which, upon hydration, spontaneously forms a cubic liquid-crystalline phase called “cubic gel”, an example of which is shown in FIG. 3. The gel can then be dispersed into nanoparticles by various means. The hallmark of cubosome morphology is a honeycomb-like structure that can be imaged using cryogenic transmission electron microscopy (cryo-TEM) as seen in FIG. 4.

Cubosomes are the subject of numerous research investigations into their use as drug delivery vehicles. However, it has now been discovered that intramammary administration of an emulsion of cubosomes, devoid of any active ingredient cargo (i.e., empty, unloaded cubosomes), to dairy cows stimulated an influx of immune cells into their mammary glands. This mechanism is believed to provide immuno-protection to the cows against, for example, mastitis-causing bacterial infection.

Thus, also provided are processes for producing mastitis treatment formulations that are suitable for use on dairy cattle and readily administered by intramammary infusion, a procedure widely utilized and requiring no new equipment or training on the part of the dairy farmer or veterinarian.

As illustrated in Example 2, a single dose of an embodiment of the present formulations prevented development of a subsequent mastitis infection in cows previously given an intramammary infusion of bacteria.

In a preferred embodiment the cubosomes are formed from the mono-glyceryl ester of oleic acid (known as “monoolein”). Monoolein is commercially available in high purity from several suppliers, primarily BASF (Ludwigshafen, Germany) under the brand name of Monomuls® 90-O 18, and is used in liquid soaps, shower/bath formulas, hair colors, and skin care products. Monoolein is a preferred reagent for use in the present formulations because it is biocompatible, bioadhesive, and digestible (Barauskas 2000). Moreover, glycerides of oleic acid including the mono-glyceride (i.e., monoolein) are naturally present in whole milk (Liu 2020).

Another equally well-characterized, cubosome-forming lipid is phytantriol, chemically named 3,7,11,15-tetramethyl-1,2,3-hexadecanetriol. Phytantriol is an ingredient in hair care products and is available from Koninklijke DSM N.V. (Royal DSM, commonly known as DSM), a Dutch multi-national company. Like monoolein, phytantriol has a hydrophilic head group connected to a long, hydrophobic hydrocarbon chain, but unlike monoolein the hydrophilic and hydrophobic moieties cannot be separated by hydrolysis and thus phytantriol represents a hydrolytically stable alternative to monoolein. Thus, in some embodiments, the cubosomes are formed from phytantriol.

Another cubosome forming substance is phosphatidyl choline, which has been studied for use in drug delivery as discussed in a publication by Xu, et al. (2018).

US Published Application No 2023/0057782 is specifically incorporated by reference herein in its entirety. In some embodiments, liquid crystal formulations described in US Published Application No 2023/0057782, e.g., Section (II) (A) are used in the disclosed methods. As introduced above, cubosome have been explored for use in drug delivery. Such cubosomes typically include one or more active agent cargos. See, e.g., FIG. 2. However, as discussed herein, empty, unloaded cubosomes on their own can have a protective effect against bacteria. This is believed to be due to an immune response induced when administered to the mammaries of the subject(s), e.g., cows or goats. Thus, typically, the provided formulations used in the disclosed methods are free from an active agent cargo. Thus, in some forms the composition consists essentially or entirely of empty, unloaded cubosomes in a pharmaceutical acceptable carrier. In some forms, the cubosomes are free from the additional substances mentioned in US Published Application No 2023/0057782, e.g., antimicrobial and/or anti-biofilm substances (see, e.g., Section (II)(B)) and mucoadhesive substances (see, e.g., Section (II)(C)). The particles can be formulated with appropriate pharmaceutically acceptable carriers into pharmaceutical compositions for administration to an individual in need thereof. The formulations are typically administered parenterally (e.g., by injection or infusion).

The particles can be formulated for parenteral administration. “Parenteral administration”, as used herein, means administration by any method other than through the digestive tract or non-invasive topical or regional routes. A preferred non-parenteral route is through the teat canal.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection.

The carrier can be a solvent or dispersion medium containing, for example, water, ethanol, one or more polyols (e.g., glycerol, propylene glycol, and liquid polyethylene glycol), and combinations thereof. The proper fluidity can be maintained, for example, by the use of a coating, such as lecithin, by the maintenance of the required particle size in the case of dispersion and/or by the use of surfactants. In some cases, isotonic agents can be included, for example, sugars or sodium chloride.

One or more pharmaceutically acceptable excipients including, but not limited to, surfactants, dispersants, emulsifiers, pH modifying agents, viscosity modifying agents, preservatives, and combination thereof can additionally or alternatively be included.

Suitable surfactants may be anionic, cationic, amphoteric or nonionic surface-active agents.

The formulation can be buffered to a pH of 3-8 for both parenteral and non-parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Formulations can be prepared using one or more pharmaceutically acceptable excipients, including diluents, preservatives, binders, lubricants, disintegrators, swelling agents, fillers, stabilizers, and combinations thereof.

Commercial intramammary (IM) mastitis treatment formulations have been provided both terminally sterilized and non-sterile. However, all current commercially available IM products are oil-based, which typically have a good shelf life. The provided formulations are typically water-based and will support microbial growth. Thus, in some forms, the formulations are sterile formulations. For example, the formulations can be radiation sterilized, e.g., by gamma irradiation. Alternatively, the formulation can be supplied as unsterilized formulations containing a preservative. Thus, the formulation can contain a preservative to prevent the growth of microorganisms. Suitable preservatives include, but are not limited to, parabens, chlorobutanol, phenol, sorbic acid, polyhexamethylene biguanide hydrochloride (PHMB), thimerosal, or combinations thereof. In some instances, the preservative is polyhexamethylene biguanide hydrochloride (PHMB). In some forms, the preservative has little or no effect on mastitis-causing bacteria upon administration of the formulation to the subject. The formulation can also be free from preservatives, for example, when sterilized by gamma irradiation. Steam sterilization is typically not used because the required temperatures can be above the melting temperature of the cubosomes.

It is believed that nanoparticle cubosomes are primarily responsible for the observed in vivo immunostimulatory effects discussed herein. Thus, the formulations typically include cubosome nanoparticles. In formulations prepared according to the methodology discussed in the experiments below, the cubosomes typically peaked in the size range of 100 to 200 nm with an average size of 137 nm. Thus, in some forms the compositions include heterodispered nanoparticles. In some forms, the compositions include homo- or heterodispered nanoparticles in the range of 50 nm to 750 nm, or 75 nm to 500 nm or 100 to 200 nm, or any other integer or fractional range or specific number between 1 nm and 1,000 nm. However, other particle sizes were also observed, and using another analytical method (i.e. single particle optical sensing) it was determined that up to about 40% of the solids volume (not the number of particles) was due to particles greater than one micron. Thus in some forms, the compositions include cubosomes that are greater than one micron. The larger particles may or may not be active, or may have the same or different (e.g., reduced) immunostimulatory activity relative to nanometer-sized particles.

Procedures for making surfactant-stabilized emulsions of phytantriol cubosomes have been described by Akhlaghi S P, et al. (2016), as referenced herein. Monoolein, like phytantriol, also forms cubosome nanoparticles upon emulsification in the presence of a surfactant as described by Saber M M, et al. (2018), referenced herein. These types of emulsions are highly preferred for use in the disclosed compositions.

To achieve a stable suspension of cubosomes and/or related nanoparticles it is typically desirable to add a surfactant to the formulation during particle formation. Suitable surfactants include, but are not limited to, block copolymers of poly(ethylene oxide) and poly(propylene oxide), for example poloxamer 407, also known as Pluronic® F127, and other such nonionic surfactants.

A preferred method of mechanical emulsification to convert cubic gel into cubosomes is the use of a rotor-stator device. Such devices are commercially available and as exemplified by the Omni Tissue Homogenizer (Omni International, Kennesaw, GA 20144).

Illustrative methods of making are also provided in the experimental examples below.

### III. Methods of Use

Methods of using the disclosed compositions for treating animals for mastitis reduction (e.g. of disease severity) or prevention are also provided. The methods typically include administering to an animal in need thereof an effective amount of a disclosed composition. In preferred embodiments, the animal is a cow, such as a dairy cow, or a population of dairy cows.

Goat milk also is an important commodity, especially outside of the USA. Mastitis in goats is frequently encountered and therefore also amenable to treatment using the disclosed compositions. Thus, in some embodiments, the animal is a goat, or a population of goats.

In some forms, the methods are dry-cow therapy or dry-goat therapy. Thus, one or more, or all, administrations of a formulation can be administered during the dry period.

Typically, the composition is effective to reduce or prevent the colonization or spread of one or more mastitis-causing bacteria. In some embodiments, the composition eliminates one or more bacteria from the mammary gland and/or other udder tissue. Mastitis-causing bacteria include Pseudomonas aeruginosa, Staphylococcus aureus, Staphylococcus epidermidis, Streptococcus agalactiae, Streptococcus uberis, Brucella melitensis, Corynebacterium bovis, Mycoplasma spp. (including Mycoplasma bovis), Escherichia coli (E. coli), Klebsiella pneumoniae, Klebsiella oxytoca, Enterobacter aerogenes, Pasteurella spp., Trueperella pyogenes (previously Arcanobacterium pyogenes), Proteus spp, Prototheca zopfii (achlorophyllic algae), and Prototheca wickerhamii.

In some forms, the subject is free-from mastitis-forming bacteria. In some forms, mastitis-causing bacteria is present in or on the subject, but the subject does not yet have mastitis. In some forms, the subject has mastitis.

Thus, the methods can be prophylactic, therapeutic, or a combination thereof.

In the experiments provided below, purposely infected cows showed some signs and symptoms of mastitis, but none became gravely ill. Each mammary was used as an individual experimental test site with rotation of test sites per cow. Thus, all cows equally received all treatments and consequently suffered some systemic effects even though one or more mammary glands might have been free of bacteria.

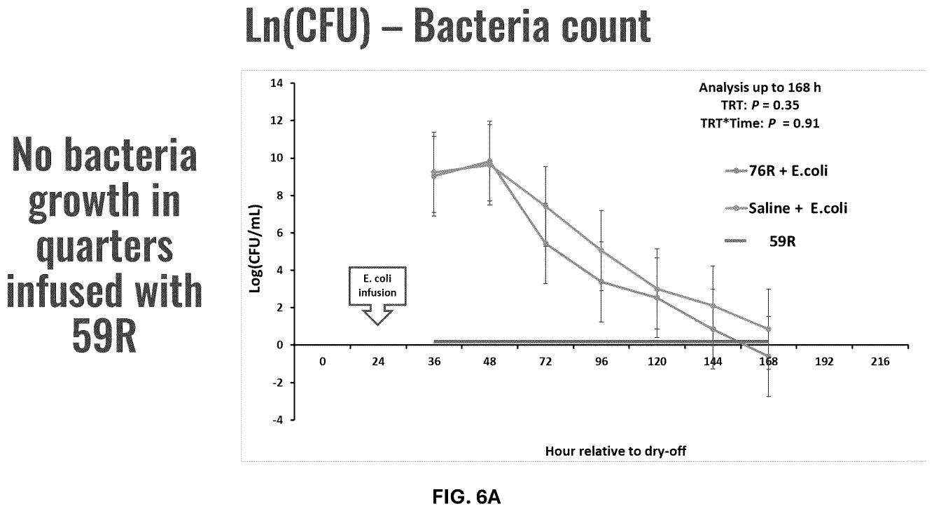

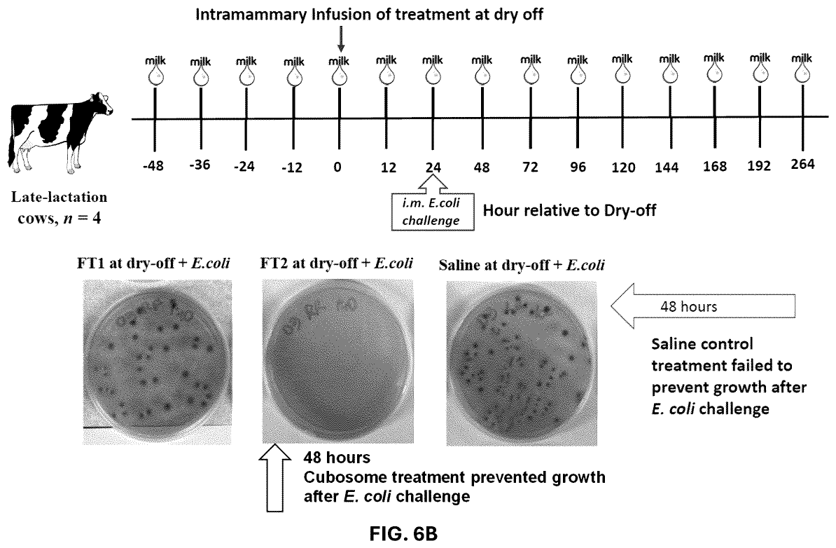

As shown in FIG. 6A, there was a dramatic prophylactic effect of 59R in preventing bacteria from surviving in treated mammary glands. In fact, not a single bacterial colony could be cultured from any 59R treated glands that were challenged. See also FIG. 6B. It is notable that the 59R formulation was prepared from only three ingredients: water, monoolein, and poloxamer 407 and did not contain a preservative. A preservative was not needed since it was sterilized by gamma irradiation. Preservatives tend to have some degree of bactericidal activity. The fact that a preservative was not present in the 59R formulation excludes the possibility of any preservative contribution to efficacy and proved that the cubosome nanoparticles alone were capable of preventing bacterial infection.

Thus, in some embodiments, the provided method results in a reduction of one or more mastitis symptoms relative to an untreated control under the same or similar conditions (e.g., exposure to bacteria, etc.). In some forms, the provided methods result in a reduction in the number of infected mammary glands and/or an increase the number of bacteria-free mammary glands relative to an untreated control under the same or similar conditions. The number of bacteria can be determined according to bacterial culture analysis such as those outlined in the examples below. In some forms, the provided method results in an increase in the number of immune cells in the mammary glands relative to an untreated control under the same or similar conditions. In some forms, the test and/or control are individual subjects or average of a population of subjects.

The compositions can be administered directly or indirectly to the mammary gland and/or other udder tissue. The compositions can be administered by infusion. The compositions can be administered via the teat canal. In particularly preferred embodiments, the compositions are administered to the mammary gland by infusion via the teat canal.

In particular embodiments, the teat canal of one or more quarters of a cow is utilized to administer treatment, for example, at the end of a lactation cycle.

Any of the disclosed methods may further include co-administration with a conventional therapy for mastitis including, but not limited, administration of one or more antibiotics. In some embodiments, the dose, frequency, or combination thereof, of antibiotic administered is lower than when administered in the absence of the disclosed compositions.

In some forms, the formulations administered to the subjects are free from an active agent cargo. Thus, in some forms the composition consists essential or entirely of unloaded cubosomes in a pharmaceutical acceptable carrier. In some forms, the cubosomes are free from the additional substances mentioned in US Published Application No 2023/0057782, e.g., antimicrobial and/or anti-biofilm substances (see, e.g., Section (II)(B)) and mucoadhesive substances (see, e.g., Section (II)(C)), and optionally the method also does not include separate administration of any other active agents. For example, in some forms, the method includes, consists essentially or completely of administering the subject(s), optionally to her/their mammary gland, e.g. through the teat cancel, unloaded, empty cubosomes. In some forms, the method, optionally repeated one, two, three, four, five, etc., times is carried one or more times, occurring partially or completely during the cow's dry off period.

In some embodiments, infected subjects such as cows or goats treated according to the disclosed methods recover from mastitis more quickly than subjects treated using conventional compositions and methods.

### EXAMPLES

### Example 1: Formulation of a Mastitis Prevention Composition

### Step 1. Cubic Gel

A bottle of monoolein was placed in a water bath at 70° C. until sufficient solid was melted. Exactly 81.00 g of the molten monoolein (Monomuls® 90-O 18) was weighted out into a beaker and 9.00 g of solid poloxomer 407 (Spectrum No. P1166) added and stirred at about 60° C. until dissolved and blended into a clear liquid. The temperature was then raised to 80° C. and the batch stirred with an overhead mixer to get a deep vortex. A disposable syringe was filled with 36 mL of distilled water and preheated to about 80° C. The hot water was then injected rapidly into the stirring melt, which caused coagulation of cubic gel. With continued stirring and heating the mixture remelted and was then allowed to cool slowly with continued stirring. Overhead stirring soon became impossible due to thickening and the stirrer was removed. Mixing was continued by hand until a taffy-like consistency was achieved. The batch was transferred to a container and allowed to cool to room temperature to obtain a yield of 115.51 g.

### Step 2. Homogenization

Exactly 60.01 g of the cubic gel was transferred from the container into a 1.25-liter glass blender jar and placed on a kitchen blender motor. Distilled water (240 mL) was added to the jar and blended at a low-speed setting for about 4 minutes to obtain a foamy white emulsion. This was transferred to a poly bottle and allowed to settle overnight at room temperature. It was then poured into a beaker and overhead mixing commenced at a moderate speed. A TH-Omni rotor-stator homogenizer was immersed in the stirring liquid such that the tip of the homogenizer probe just cleared the overhead mixing blade. Stirring was increased to obtain a deep vortex, and the homogenizer started and let run at maximum speed for 10 minutes.

### Step 3 Packaging

Standard 10cc, unit-dose, disposable, opaque white udder injector syringes were filled with the above emulsion. This was accomplished using a 60cc syringe of the emulsion and forcing the contents into the 10cc syringes via the soft cannula tip, which caused the udder injector plunger to move backwards. The filled & capped udder injector syringes were placed in Zip-Loc pouches and put in shipping boxes.

### Step 4 Sterilization

The boxes were shipped to Sterigenics (Corona, CA) and gamma irradiated at a dose of 25kGy to ensure sterility.

### Step 5 Analysis

The radiation sterilized samples are identified by lot no. 008-59R (59R hereinafter). The cubosomes contained an amphiphilic lipid and a stabilizing agent that, when hydrated, spontaneously organize into a cubic liquid-crystalline phase referred to as “cubic gel” (FIG. 3). This gel can subsequently be broken down into nanoparticles using various dispersion techniques. Samples of 59R were submitted for cryo-TEM imaging, which confirmed the presence of cubosomes (FIG. 4). As shown in FIG. 4, the cubosomes had a honey-comb like internal structure. Additional samples were sent for nanoparticle tracking analysis, which showed a mean particle size of 125 nm and a concentration of 1.76×10¹² nanoparticles per mL.

### Example 2: Evaluation of Mastitis Prevention in Dairy Cows at Dry-Off

### Materials and Methods

To determine the effectiveness the formulation, a study was designed to simulate conditions in which bacteria get introduced into the cow's mammary glands at the time when they are least able to fight the germs, namely at dry-off. In order not to induce a fatal infection a relatively mild dose of E. coli was used in this study with the expectation that all the animals would get sick but could recover in a few days without needing antibiotics. Thus, the intramammary treatment was given first as a prophylactic and 24 hours later they received the intramammary dose of bacteria immediately after the last full milking (i.e. dry-off). The volume of milk collected after dry-off was just enough for testing and did not count as milking.

Four lactating Holstein cows in their mid-to late lactation were selected to perform intramammary E. coli challenge. All cows were healthy, had milk SCC below 200,000 cell/mL, no incidence of mastitis in the last 3 months, and produced more than 15 kg of milk per day. All cows were housed in the same free-stall barn bedded with sand. Cows were milked using a portable milker twice a day (0700 and 1900 h) and fed once a day.

Individual milk samples from all quarters of each cow were aseptically collected two days before the challenge. Samples were used to measure SCC (<200,000 cells/mL) and placed on blood agar plates to confirm the absence of detectable bacterial growth.

The bacterial challenge used was E. coli 727. It was sub-cultured on trypticase soy agar to achieve a pure clonal colony. The colony was expanded in Brain-Heart Infusion (BHI) broth to about 10¹¹ cfu per mL and frozen in BHI containing 15% sterile glycerol at −80° C. in 0.5 mL portions. The pure culture was confirmed by testing two portions for uniformity of growth and proper reaction on coliform selective agar.

To prepare a challenge dose of 20 cfu/mL of the E. coli, one vial of bacteria was thawed and placed in 10 mL of BHI. The culture was incubated overnight at 37° C. with shaking. In the morning, the culture was centrifuged at 1500×g for 10 minutes. The medium was removed and a suspension of 50 mL prepared in sterile phosphate buffered saline (PBS, pH 7.4). The density of the culture was adjusted to 1.0 OD using a spectrophotometer by dilution with PBS. This density represents a culture at 2×10⁸ cfu/mL. The culture was diluted in 100-fold steps to a density of 20 cfu/mL in PBS. It was held on ice until ready to use. During and after the intramammary E. coli challenge, all cows remained in the same pen. All cows received a shot of oxytocin to ensure complete milking before bacteria infusion. Immediately after morning milking (0700 h), all quarters were cleaned and disinfected with 70% ethanol.

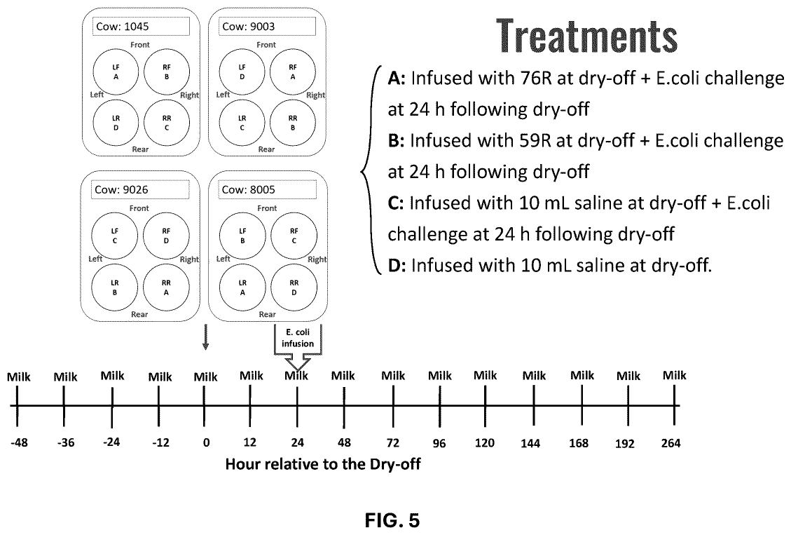

After the last milking, quarters within each cow were alternately infused with 10 mL of FT1 (i.e., 76R), FT2 (i.e., 59R), or saline (SAL, 2 quarters). At 24 h post-dry-off, 10 mL of E. coli 727 suspension (360 CFU/mL) was intramammarily infused into quarters that had received FT1, FT2, or SAL, while the remaining SAL quarter did not receive E. coli and served as a control. As shown on the treatment plan in FIG. 5, for each cow, one quarter (C) was infused with 1 mL saline followed by 20 cfu E. coli preparation, the second quarter (A) infused with 76R (an unrelated experimental test material) followed by 20 cfu E. coli preparation, the third quarter (B) infused with 1 mL saline and 20 cfu E. coli preparation following by 59R treatment starting at 12 h later, the last quarter (D) infused with 1 mL saline as control. The teats were massaged throughout to move the inoculum into gland cistern. The administration was performed after milk sample collection and milking.

Quarter milk or mammary secretion samples were collected every 12 h from −48 to 264 h relative to dry-off for composition analysis, and E. coli enumeration was performed at 36, 48, 72, 96, 120, 144, and 168 h post-dry-off.

Blood was drawn from coccygeal vessels of all cows at −1, 6, 12, 18, 24, 48, 72, 96, 120, 144, 168, 216, and 264 h relative to the bacterial infusion for collection of serum and plasma for acute phase protein measures. Additional blood samples were collected at the same time in one EDTA vacutainer (Becton Dickinson) to assess a hematologic profile (University of Georgia Veterinary Diagnostic Laboratory, Athens, GA) using an ADVIA 2120i Hematology System (Siemens, Tarry town, NY).

Aseptic milk samples from all quarters were collected at −1, 6, 12, 18, 24, 48, 72, 96, 120, 144 168, 192, 216, 240 and 264 h relative to the bacterial infusion.

All quarter samples were collected following a careful cleaning of the teat end, with 70% ethanol final preparation. The teat was stripped three times, then the milk sample collected for bacterial culture, milk composition, total and differential count of somatic cells, and fat and skim milk collection. The skim milk was used to measure pro- and anti-inflammatory cytokines (TNF-α, IL10, IL8), acute phase proteins (haptoglobin and milk amyloid A) and bovine serum albumin.

### Results

All cows showed some signs and symptoms of mastitis, but none became gravely ill. Each mammary was used as an individual experimental test site with rotation of test sites per cow. Thus, all cows equally received all treatments and consequently suffered some systemic effects even though one or more mammary glands might have been free of bacteria.

As shown in FIGS. 6A and 6B (bottom panel), E. coli counts in the 76R+E. coli and saline+E. coli quarters peaked at approximately 48 h post-challenge, reaching levels of ˜17,000 CFU/mL, and then gradually declined to ˜120 CFU/mL by 120 h over the subsequent days. In contrast, there was a dramatic prophylactic effect of 59R in preventing bacteria from surviving in treated mammary glands (FIGS. 6A and 6B. In fact, not a single bacterial colony could be cultured from any 59R treated glands that were challenged (FIGS. 6A and 6B).

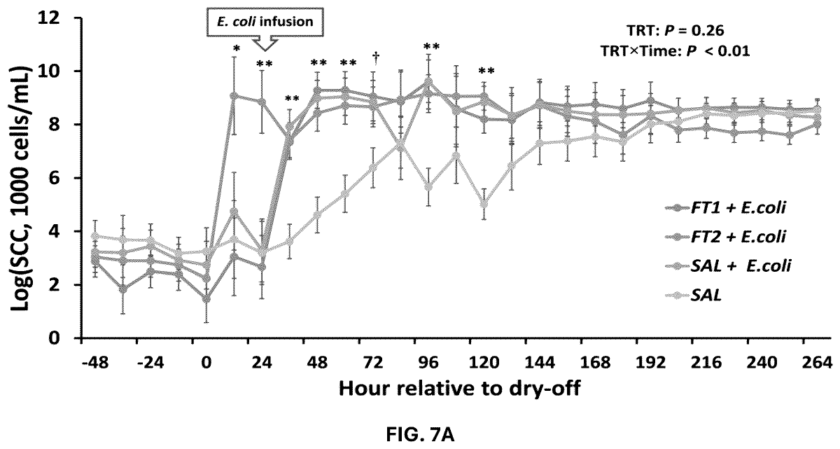

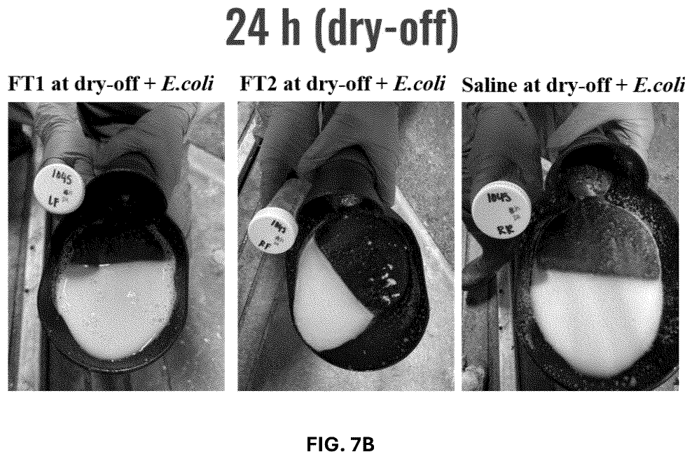

As shown in FIGS. 7A and 7B, quarters treated with FT2 (i.e., 59R) demonstrated an increase in milk somatic cell count (SCC) prior to the E. coli challenge. Following E. coli infusion, all challenged quarters, regardless of treatment, showed elevated SCC between ˜36 and ˜120 h post-dry-off, with FT1- and FT2-treated quarters (i.e., 76R- and 59R-treated quarters) reaching similar peak levels (FIG. 7A).

### CONCLUSION

The 59R formulation (FT2) demonstrated increased efficacy compared with the 76R formulation (FT1) in stimulating immune cell infiltration into the mammary gland and in eliminating bacterial infection during the early dry period. These findings suggest that cubosome-based formulations, particularly 59R, hold significant potential as a dry cow therapy for preventing bacterial infections during this high-risk stage.

All references, including references cited therein and the teachings thereof are incorporated by reference herein in their entireties.

### BIBLIOGRAPHY

Akbar S, Anwar A, Ayish A, Elliott J M, and Squires A M, “Phytantriol based smart nano-carriers for drug delivery applications”, European Journal of Pharmaceutical Sciences, 101, 31 (2017).
Akhlaghi S P, Ribeiro I R, Boyd B J, and Loh W, “Impact of preparation method and variables on the internal structure, morphology, and presence of liposomes in phytantriol-Pluronic®-127 cubosomes”, Colloids and Surfaces B: Biointerfaces, 145, 845 (2016).
Ameen F, et al., “Prevalence of antibiotic resistant pathogens in dairy cows in Egypt and potential biological control agents from plant endophytic actinobacteria”, Saudi Journal of Biological Sciences, 26, 1492-1498 (2019).
Bradley A J and Green M J, “The importance of the nonlactating period in the epidemiology of intramammary infection and strategies for prevention”, Vet Clin North Am Food Anim Pract. 20:547-568 (2004).
Bhosale R R, Osmani, R A, Harkare, B R, Ghodake, P P (2013) Cubosomes: The Inimitable Nanoparticulate Drug Carriers, Sch. Acad. J. Pharm., 2 (6): 481-486
Barauskas J, Johnsson M, Tiberg F (2000) Self-assembled lipid superstructures: beyond vesicles and liposomes. Nano Lett. 5 (8): 1615-1619.
Dorgan R J J, “Veterinary compositions for treating mastitis”, US Patent Publication US2016/0030398, Feb. 4, 2016.
Dyett B P, Yu H, Sarkar S, Strachan J B, Drummond C J, and Conn C E (2021) Uptake Dynamics of Cubosome Nanocarriers at Bacterial Surfaces and the Routes for Cargo Internalization, ACS Applied Materials & Interfaces 13 (45), 53530-53540
Hozyen H F, et al., “Enhanced antibacterial activity of capped zinc oxide nanoparticles: A step towards the control of clinical bovine mastitis”, Veterinary World, 12:8, 1225-1223 (2019).
Lai X, Han M-L, Ding Y, Chow S H, Le Brun A P, Wu C-M, Bergen P J, Jiang J, Hsu H-Y, Muir B W, White J, Song J, Li J, and Shen H-H (2022) A polytherapy based approach to combat antimicrobial resistance using cubosomes, Nature Communications, 13:343.
Liu Z, Li C, Pryce J, and Rochfort S (2020) Comprehensive Characterization of Bovine Milk Lipids: Triglycerides, ACS Omega, 5, 12573-12582.
Lopez C, Carrasco M, Gallardo P, Rademacher H, and Gomez G, “Natural antiseptic solution to be used as teat dip (dipping) during milking for prevention and control of mastitis”, WO 2014/053977, Apr. 10, 2014.
McLain V C, “Final report on the safety assessment of phytantriol”, International Journal of Toxicology, 26 (Suppl. 1): 107-114 (2007).
Milak S and Zimmer A, “Glycerol monooleate liquid crystalline phases used in drug delivery systems”, International Journal of Pharmaceutics. 478:2, 569 (2015).
Paudyal S, et al., “Relationships among quarter milk leukocyte proportions and cow and quarter-level variables under different intramammary infection statuses”, Transl Anim Sci, 2, 231-240 (2018).
Peralta O A, et al., “Safety and efficacy of a mesenchymal stem cell intramammary therapy in dairy cows with experimentally induced Staphylococcus aureus clinical mastitis”, Scientific Reports, 10, 2841 (2020).
Ruegg P L (2021) What Is Success? A Narrative Review of Research Evaluating Outcomes of Antibiotics Used for Treatment of Clinical Mastitis. Front. Vet. Sci. 8:639641. doi: 10.3389/fvets.2021.639641
Saber, et al., “Targeting colorectal cancer cell metabolism through development of cisplatin and metformin nano-cubosomes”, BMC Cancer (2018) 18:822 https://doi.org/10.1186/s12885-018-4727-5
Sivadasan D, Sultan M H, Alqahtani S S, and Shamama-Javed S (2023) Cubosomes in Drug Delivery—A Comprehensive Review on Its Structural Components, Preparation Techniques and Therapeutic Applications. Biomedicines, 11, 1114.
U.S. Centers for Disease Control and Prevention (2019) Antibiotic Resistance Threats in the United States. www.cdc.gov/DrugResistance/Biggest-Threats.html.
Xu Y, et al., “Characterization of a Liquid Crystal System for Sustained Release of a Peptide BMS-686117”, AAPS Pharm Sci Tech, 19 (1), 348 (2018).

While the invention has been illustrated by a description of embodiments described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative examples shown and described. Accordingly, departures may be made from such details without departing from the spirit or scope of applicants' general inventive concept.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. A composition for the reduction in severity or prevention of mastitis comprising an emulsion of cubosomes.

2. The composition of claim 1, wherein the cubosomes are empty, unloaded cubosomes.

3. The composition of claim 2, wherein the composition comprises, or consists essentially of, or consists of the empty, unloaded cubosomes and a pharmaceutically acceptable carrier.

4. The composition of claim 2, wherein the pharmaceutically acceptable carrier further comprises a preservative.

5. The composition of claim 4, wherein the preservative is selected from parabens, chlorobutanol, phenol, sorbic acid, polyhexamethylene biguanide hydrochloride (PHMB), thimerosal, and combinations thereof.

6. The composition of claim 2, wherein the empty, unloaded cubosomes have immunostimulatory activity.

7. The composition of claim 6, wherein the immunostimulatory activity comprises inducing migration of immune cells toward the composition.

8. The composition of claim 2, wherein the empty, unloaded cubosomes are nanoparticles.

9. The composition of claim 8, wherein the nanoparticles comprise, or consist essentially of, or consist of a homogeneous or heterogenous dispersion of the nanoparticles having a size in a range of 50 nm to 750 nm, or 75 nm to 500 nm, or 100 to 200 nm, or any other integer or fractional range or specific number between 1 nm and 1,000 nm.

10. The composition of claim 2, wherein the empty, unloaded cubosomes further comprise an amphiphilic lipid and a surfactant.

11. The composition of claim 10, wherein the surfactant is poloxamer 407 and the amphiphilic lipid is selected from monoolein, phytantriol, and phosphatidyl choline.

12. The composition of claim 10, wherein the amphiphilic lipid is monoolein.

13. The composition of claim 10, wherein the amphiphilic lipid is phytantriol.

14. The composition of claim 10, wherein the amphiphilic lipid is phosphatidyl choline.

15. A method for reduction of severity or prevention of mastitis comprising administering the animal an effective amount of the composition of claim 2.

16. The method of claim 15, wherein the animal is a cow or a goat; optionally wherein the cow is a dairy cow.

17. The method of claim 15, wherein the composition is administered directly or indirectly to the mammary gland.

18. The method of claim 15, wherein the composition is administered by injection or infusion.

19. The method of claim 15, wherein the composition is administered via the teat canal.

20. A method of preventing mastitis in a dairy cow or goat by infusing a composition of claim 2 into the mammary gland of the cow or goat via the teat canal.
